(19) United States
(12) Patent Application Publication
(10) Pub. No.: US 2021/0393991 A1
(43) Pub. Date: Dec. 23, 2021

(54) SYSTEMS AND METHODS FOR TRANSCRANIAL BRAIN STIMULATION USING ULTRASOUND

(71) Applicant: X Development LLC, Mountain View, CA (US)

(72) Inventors: Vladimir Miskovic, Binghamton, NY (US); Thomas Peter Hunt, Oakland, CA (US); Matthew Dixon Eisaman, Port Jefferson, NY (US)

(21) Appl. No.: 17/350,542

(22) Filed: Jun. 17, 2021

Related U.S. Application Data
(60) Provisional application No. 63/129,971, filed on Dec. 23, 2020.
(60) Provisional application No. 63/041,257, filed on Jun. 19, 2020.

(51) Int. Cl. A61N 7/00

(52) U.S. Cl. CPC A61N 7/00; A61N 2007/0056; A61N 2007/0026

(57) ABSTRACT

A transcranial ultrasonic stimulation headset includes a coupling system, one or more ultrasound transducers configured to generate and direct a first focused ultrasound beam at a region within a portion of a subject's brain, one or more sensors configured to measure a response from the portion of the subject's brain in response to the first focused ultrasound beam, and an electronic controller in communication with the one or more ultrasound transducers configured to dynamically adjust, based on the measured response from the portion of the subject's brain, a particular stimulation parameter for the one or more ultrasound transducers to generate and direct a second focused ultrasound beam at the region within a portion of the subject's brain.

### CROSS REFERENCE TO RELATED APPLICATION

This application claims the benefit of U.S. Provisional Application No. 63/129,971, filed Dec. 23, 2020, and this application also claims the benefit of U.S. Provisional Application No. 63/041,257, filed Jun. 19, 2020, the contents of each are incorporated by reference herein.

### FIELD

This specification relates to transcranial brain stimulation.

### BACKGROUND

Stimulation of the brain in humans is typically performed using electrical or magnetic fields without feedback and with respect to a generic position relative to a subject's head, and typically is not based on measurements of the particular subject's brain activity or tailored to the particular subject's brain morphology or cranial structure.

### SUMMARY

Brain stimulation is used to treat movement disorders as well as disorders of affect and consciousness. There is growing evidence that stimulation can improve memory or modulate attention and mindfulness. Additional therapeutic applications include rehabilitation and pain management.

The methods described here perform transcranial stimulation of large-scale brain networks in real-time and adjust the stimulation based on brain activity patterns detected in response to the stimulation. In particular, the methods allow for transcranial stimulation based on brain activity, skull structure, tissue displacement, and other physical features specific to a particular subject, all of which can vary between subjects and affect where and how a brain stimulation should be applied to the subject. The stimulation is adapted both to a subject's natural brain activity patterns and to the complexity of such patterns.

Computer models, including machine learning models can analyze a measured response to transcranial stimulation and generate stimulation parameters. For example, brain activity and function measurements can be used with statistical and/or machine learning models to determine a current brain state, to analyze the subject's physical and neurological response to stimulation, and to determine future stimulation parameters, among other processes. In some cases, the models can be applied to the method to quantify the effectiveness of a particular set of stimulation parameters. The methods can use additional biomarker inputs to determine the stimulation parameters or classify feedback. For example, the methods can use vital signs of the subject or verbal feedback from the subject as additional input to the model to improve the accuracy of the model and to personalize the models and stimulation to the subject.

Systems for implementing the methods can be embodied in various form factors. In some implementations, the system includes a brain stimulation headset or helmet. In other implementations, the system includes a set of headphones or goggles.

In general, one innovative aspect of the subject matter described in this specification may be embodied in a transcranial system transcranial ultrasonic stimulation headset that includes a coupling system, one or more ultrasound transducers configured to generate and direct a first focused ultrasound beam at a region within a portion of a subject's brain, one or more sensors configured to measure a response from the portion of the subject's brain in response to the first focused ultrasound beam, and an electronic controller in communication with the one or more ultrasound transducers configured to dynamically adjust, based on the measured response from the portion of the subject's brain, a particular stimulation parameter for the one or more ultrasound transducers to generate and direct a second focused ultrasound beam at the region within a portion of the subject's brain.

In some implementations, the system includes an insert having a particular material property, and the electronic controller is further configured to adjust the particular material property of the insert based on the adjusted stimulation parameter. In some implementations, the insert is shaped based on a physical structure of the subject's skull.

In some implementations, dynamically adjusting the stimulation parameter is performed based on the subject's verbal feedback.

In some implementations, the coupling system comprises a cooling system having cooling fluid, and the coupling system is integrated with a neck support structure. In some implementations, the cooling fluid has a material property, and the electronic controller is further configured to adjust the material property of the insert based on the adjusted stimulation parameter. In some implementations, the material property is a density.

In some implementations, the electronic controller is further configured to dynamically adjust two more stimulation parameters for the one or more ultrasound transducers.

In general, another innovative aspect of the subject matter described in this specification may be embodied in a method for transcranial stimulation including directing, by one or more ultrasound transducers, a first focused ultrasound beam at a region within a portion of a subject's brain, measuring, by one or more sensors and in response to the first focused ultrasound beam, a response from the subject's brain, and dynamically adjusting, based on the measured response from the portion of the subject's brain and by a controller, a stimulation parameter for the one or more ultrasound transducers to generate and direct a second focused ultrasound beam at the region within the portion of the subject's brain.

In some implementations, the method includes adjusting, by the controller and based on the adjusted stimulation parameter, a particular material property of an insert of a coupling system communicatively connected to the controller.

In some implementations, measuring a response from the subject's brain includes identifying an activity pattern of a subject's brain and determining, based on the identified activity pattern of the subject's brain and a target parameter, one or more stimulation parameters. In some implementations, the target parameter is a selected set of one or more physiological measurements of the subject. In some implementations, the target parameter is determined based on the subject's verbal feedback.

In some implementations, dynamically adjusting the particular stimulation parameter is performed based on the subject's verbal feedback.

In some implementations, dynamically adjusting the particular stimulation parameter comprises using machine learning techniques to generate one or more adjusted stimulation parameters.

In some implementations, dynamically adjusting the particular stimulation parameter comprises adjusting two more stimulation parameters for the one or more ultrasound transducers.

Other embodiments of this aspect include corresponding systems, apparatus, and computer programs, configured to perform the actions of the methods, encoded on computer storage devices.

The details of one or more implementations are set forth in the accompanying drawings and the description, below. Other potential features and advantages of the disclosure will be apparent from the description and drawings, and from the claims.

### BRIEF DESCRIPTION OF THE DRAWINGS

FIG. 1 is a diagram of an example configuration of a transcranial brain stimulation system.

FIG. 2 is a diagram of an example machine learning process for transcranial brain stimulation.

FIGS. 3A, 3B, 3C, and 3D are illustrations of example form factors of the transcranial brain stimulation system.

FIG. 4 is a flow chart of an example process of transcranial brain stimulation.

Like reference numbers and designations in the various drawings indicate like elements. The components shown here, their connections and relationships, and their functions, are meant to be examples only, and are not meant to limit the implementations described and/or claimed in this document.

### DETAILED DESCRIPTION

Stimulation of particular regions of a brain, including large-scale brain networks—various sets of synchronized brain areas linked together by brain function—can be used to treat neurological and psychiatric disorders and certain effects of physical disorders. The methods and systems described here can be used for therapeutic purposes to treat psychiatric conditions such as anxiety disorders, trauma and stressor-related disorders, panic disorders, and mood disorders as well as treating the physical symptoms of various disorders, diseases, and conditions. For example, the described system can be used to treat phobias, reduce anxiety, and/or control tremors or tinnitus, among other applications. Additionally, these methods can be used for cognitive remediation (e.g., improve or restore executive control), to improve alertness, and/or to aid sleep regulation, among other applications.

These methods can also be used to produce positive effects on a subject's memory, attention, and focus. For example, the described method can be used to produce a desired psychological state in a subject, to aid in meditation, to increase focus, and/or to enhance learning and skill acquisition, among other applications.

Brain stimulation methods generally are not personalized for particular subjects and their needs, and do not take into account skull structure or brain activity that occurs in response to the stimulation. These methods typically are not tailored to a particular subject's brain morphology or activity and such stimulation waveforms are often highly artificial (e.g., a square wave or random noise), without resembling natural patterns of brain activity.

The described methods and systems perform transcranial stimulation of the brain, allow for stimulation of large-scale brain networks in real-time, and adjust the stimulation parameters, including frequency, power, focal length, time duration, and spot size, based on measurements taken of the subject's brain structure and activity patterns and cranial structure and the surrounding tissue, hair, and other biomaterial. These measurements can be used with statistical and/or machine learning models to determine a current brain state, to analyze the subject's response to the stimulation, and to determine future stimulation parameters. In some implementations, the measurements can be used to map out cranial and brain structure, connectivity, and functionality to personalize stimulation to a particular subject.

For example, the described methods can include providing ultrasonic stimulation according to a particular set of stimulation parameters to a particular area of a subject's brain, contemporaneously or near-contemporaneously recording brain activity detected by sensors, adjusting stimulation parameters based on the detected brain activity, and applying the adjusted stimulation parameters.

The described methods and systems can be implemented automatically (e.g., without direct human control). For example, the controller can automatically detect and identify activity of a particular subject's brain and use the activity to tailor stimulation parameters and detection techniques to the particular subject's brain.

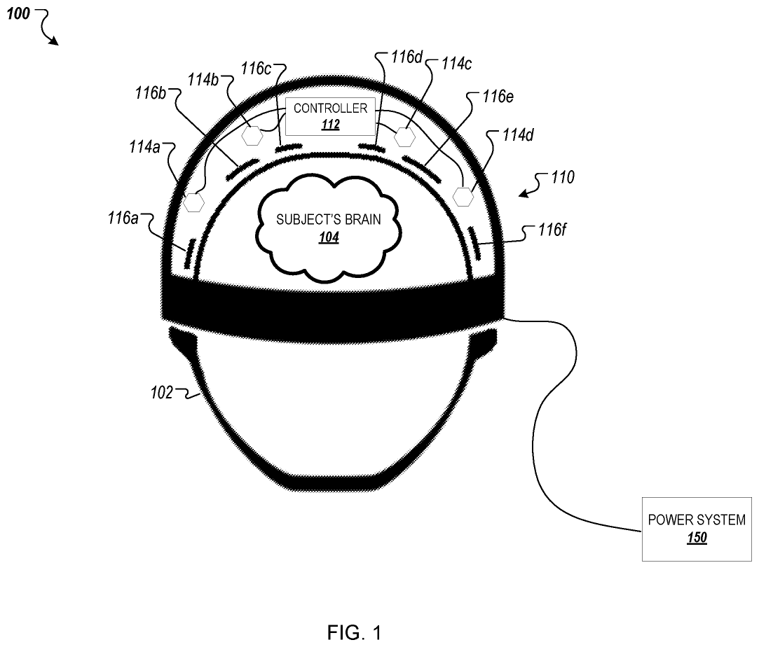

FIG. 1 is a diagram of an example configuration 100 of a transcranial brain stimulation system 110. System 110 provides transcranial stimulation of large-scale brain networks. For example, system 110 can be used to stimulate a target area of a subject's brain and, based on measured brain activity, the system 110 can adjust various parameters of the stimulation of the target area.

System 110 is a transcranial ultrasonic stimulation system that includes a coupling system that improves and/or facilitates coupling between the subject and one or more ultrasound transducers that are configured, before and/or during use, to generate and direct a first focused ultrasound beam at a region within a portion of a subject's brain. The system also includes one or more sensors configured, during use, to measure a response from the portion of the subject's brain in response to the first focused ultrasound beam as well as measured feedback from the subject or stimulation beam. The system includes an electronic controller in communication with the at least two ultrasound transducers configured, during use, to dynamically adjust, based on the measured response from the portion of the subject's brain, a stimulation parameter for the one or more ultrasound transducers to generate and direct a second focused ultrasound beam at the region within a portion of the subject's brain.

System 110 provides a high degree of control over stimulation parameters and patterns. System 110 can provide transcranial stimulation by controlling the parameters of pulsed ultrasonic waves or an ultrasound beam. Different stimulation parameters and forms can produce different effects on subject behavior and on the brain. For example, constant stimulation, alternating stimulation, and random noise stimulation can produce different resulting behavior. System 110 can provide direct stimulation of cortexes of the brain. For example, system 110 can be used to directly stimulate the visual cortex, the auditory cortex, or the somatosensory cortex through ultrasonic stimulation. The methods can also be applied to stimulate peripheral nerves, such as the vagus nerve.

In some implementations system 110 can provide transcranial stimulation by modulating electrical signals to produce constant waveforms, alternating waveforms, and random noise signals. Constant waveform stimulation can be performed by providing constant direct current (DC) transcranially using conductors to provide constant voltage. Alternating waveform stimulation can be performed by providing sine waves transcranially at different frequencies. Random noise stimulation can be performed by providing random electrical signals transcranially, for example, that imitate white noise patterns or pink noise patterns. Different stimulation patterns can produce different effects in behavior and on the brain. For example, constant waveform stimulation, alternating waveform stimulation, and random noise signal stimulation can produce different resulting behavior. Colored noise patterns, in particular, can be designed to match the statistical properties of natural brain signals as observed across different states of arousal (e.g., drowsiness versus alert attention or cognitive engagement).

In this particular example, system 110 includes a wearable headpiece that can be placed on or around a subject's head or neck. In some implementations, system 110 can include a network of individual transducers and sensors that can be placed on the subject's head or a system that holds individual transducers and sensors in fixed positions around the subject's head.

In this particular example, system 110 can be used without an external power source. For example, system 110 can include an internal power source. The internal power source can be rechargeable and/or replaceable. For example, system 110 can include a replaceable, rechargeable battery pack that provides power to the transducers and sensors.

Subject 102 is a human subject of transcranial stimulation.

A focal spot, or target area, within subject's brain 104 can be targeted. The target area can be, for example, a specific large-scale brain network associated with a particular state of a subject's brain 104. In some implementations, the target area can be automatically selected based on detection data. For example, the system 110 can adjust the targeted area within subject's brain 104 based on detected brain activity. In some implementations, the target area can be selected manually based on a target reaction from subject's brain 104 or a target reaction from other body parts of the subject. In some implementations, system 110 can stimulate peripheral nerves in addition to brain regions. For example, system 110 can stimulate peripheral nerves such as the vagus nerve to treat affective disorders such as depression or anxiety.

Transcranial stimulation system 110 is shown to include a controller 112, sensors 114a, 114b, and 114c (collectively referred to as sensors 114 or sensing system 114), and transducers 116a, 116b, 116c, 116d, 116e, 116f, 116g, and 116h (collectively referred to as transducers 116 or stimulation generation system 116). System 110 is configured to provide transcranial stimulation of large-scale brain networks through use of one or more transducers. For example, system 110 can include a single transducer 116. The one or more transducers 116 provide ultrasound stimulation, and can be controlled separately or in groups of two or more transducers 116. In some implementations, one or more transducers 116 can provide electrical or magnetic stimulation.

System 110 can uses low intensity, pulsed ultrasonic stimulation to stimulate a target area of subject's brain 104. In some implementations, system 110 uses high intensity stimulation subject to thresholds as monitored by system 110 for the subject 102's safety as described in further detail below.

Stimulation generation system 116 can include multiple elements and types of transducers 116. Stimulation generation system 116 can include one or more patterns and arrangements of arrays of transducers 116. For example, stimulation generation system 116 can include multiple transducers 116 that can target multiple areas, and allow system 116 to target different locations. If, for example, stimulation generation system 116 operates according to a Cartesian coordinate system, the multiple transducers 116 that can be arranged in arrays allow system 110 to dynamically target areas and move the target area in the X, Y, and Z directions. Stimulation generation system 116 can use phased arrays that can target multiple areas of different depths. The phased arrays allow stimulation generation system 116 to generate and transmit pulsed emissions that have additive effects.

In some implementations, stimulation generation system 116 can include dedicated transducers 116 that target particular beam focal locations. For example, stimulation generation system 116 can include one or more transducers 116 that are arranged specifically to target a particular area of subject's brain 104.

Stimulation generation system 116 can include components that enable the system 110 to generate, direct, and focus emissions, including components such as delay lines or zone plates. For example, stimulation generation system 116 can include delay lines that are arranged specifically for particular transducers 116 and/or particular focal locations within subject 102.

In some implementations, multiple stimulation generation systems or arrays of transducers are operated by the system 110 in order to stimulate multiple areas of subject 102. For example, multiple stimulation generation systems can include multiple types of transducers having different specifications and capabilities can be operated in order to stimulate multiple areas of subject's brain 104.

The type of stimulation and the areas of a brain that can be stimulated are closely related to, and in some cases, governed by, the modality with which the stimulation is provided. As discussed above, transducers 116 can provide electrical, magnetic, and/or ultrasound stimulation. If, for example, controller 112 applies focused ultrasound stimulation, controller 112 would need to focus and steer a wide bandwidth of the ultrasound beam into a target region.

System 110's use of ultrasonic stimulation provides greatly improved spatial resolution (millimeter or sub-millimeter resolution) as compared to methods that use electrical or magnetic stimulation (on the order of centimeters). System 110 can target multiple regions using multiple acoustic beams and interference between the beams to produce stimulation according to desired stimulation parameters.

Ultrasound stimulation can target shallow or deep tissue and provides resolution on the order of millimeters. With finer resolution, controller 112 can target deep brain structures such as basal ganglia. For example, controller 112 can use ultrasound stimulation to control tremors by detecting the frequency of a tremor, classifying the frequency as a certain color of noise, and applying stimulation to shift the color of noise.

In some implementations, electrical stimulation may provide a coarser resolution than ultrasound stimulation. Electrical stimulation can be applied using, for example, high-definition electrodes that can be used to target regions such as the frontal cortex of a subject's brain to produce cognitive effects.

In addition to controlling the intensity and shape of stimulation signals, controller 112 can control the time scale of signal switching. In some implementations, the switching frequency is lower than that used in focused ultrasound. In some implementations, the switching frequency is adapted based on a subject's natural brain activity pattern frequencies.

Controller 112 implements safety measurements to ensure the proper use of system 110. Controller 112 can monitor the emissions from transducers 116 and the subject 102's biological response to the emissions. Controller 112 can receive data from sensors 114 and other sensing systems communicatively connected to the system 110 and use the data to improve the stimulation of subject 102. Controller 112 can also receive data measuring the emissions from subject 102 to monitor the usage of the system 110.

In some implementations, controller 112 monitors the local speed of sound using the ultrasonic pulses emitted. For example, controller 112 can monitor reflections of the ultrasonic emissions from subject 102 to estimate the local speed of sound at the subject 102's body. The speed of sound propagation is dependent on the density of the material from which the sound waves are reflected, and thus is correlated with temperature. This estimation can be used relative to a baseline measurement for a particular subject 102 and used by controller 112 to monitor heat levels at the subject 102's skull and head to adjust stimulation. Controller 112 can, for example, determine the local speed of sound at a “cold start,” when stimulation begins, and determine the local speed of sound at a later time, calculating a difference in the amount of time that it takes for the reflected wave to return and thus a change in temperature. Controller 112 can determine, based on a change in the local speed of sound, that the levels of heat being generated from the present stimulation of subject 102 is too high, and can adjust the stimulation by reducing the intensity, stopping the stimulation, etc. for subject 102's safety. In some implementations, controller 112 can continue to monitor the local speed of sound to determine whether to begin stimulation again and/or at what levels the stimulation should be performed.

Controller 112 can also monitor the heat emissions from subject 102 directly. For example, controller 112 can receive sensor data indicating the subject 102's skin temperature local to the target area being stimulated and adjust emissions to the subject 102 to keep the level of heat generated from stimulation to a safe level. In some implementations, controller 112 can measure the reflection from the ultrasonic emissions. Controller 112 can use these reflection measurements to monitor heat levels. For example, controller 112 can use reflection measurements to determine the intensity and timing of the reflections to determine the amount of energy that is currently or cumulatively absorbed by the subject 102. Sustained levels of high intensity emissions can cause injury and/or generate too much heat; controller 112 can adjust stimulation generated by system 110 to control the total thermal dose delivered to the subject 102's scalp or skull.

Controller 112 can calculate the appropriate phases for therapeutic ultrasound beams that have been steered to the target area of subject's brain 104. These phases can interact to increase or decrease resolution and/or power, and can be calculated automatically using various algorithms, including machine learning algorithms as described above. Controller 112 can automatically determine appropriate phases by changing phases for the ultrasonic output of transducers 116 and use an amount of power returned from the target area to determine whether to change the pressure or phase of each transducer. For example, controller 112 can use the amount of power returned from the target area of subject's brain 104 being stimulated by ultrasonic pulses, and automatically determine a change to the power level of the ultrasound stimulation. Controller 112 can use, for example, phased arrays that emit ultrasound pulses and adjust the phases of these pulses for maximum intensity, up to a predetermined safety threshold level.

In some implementations, there is a hologram of the focal spot of the ultrasound beam that is used for beamforming. The hologram is an acoustic holographic beam that shapes the ultrasound. The projection of the focal spot can be the location of the target area of subject's brain 104. Controller 112 can use a signal processing technique with transducers 116 for beamforming. Controller 112 can provide directional signal transmission or reception through beamforming by combining elements in an antenna array such that signals at particular angles experience constructive interference while others experience destructive interference in order to achieve spatial selectivity. Based on the ultrasound imaging or measurements, system 110 can match propagation delays to the target from each element in the phased array. For example, the array can be one-dimensional or multi-dimensional, and can be controlled such that the ultrasound waves arrive at the target in-phase and in-focus. The directional transmission and focus process is controlled through a technique similar to phase reconstruction for imaging techniques, but with the specific aim of maximizing delivered energy to the target through complex media without homogeneous propagation properties.

System 110 can stimulate target areas of different shapes. For example, system 110 can provide an elongated focus that is not circular. Controller 112 can control transducers 116 to stimulate target areas of different shapes by, for example, steering individual transducers 116 and/or an array of transducers 116. System 110 can stimulate target areas of rectangular, oblong, linear, and triangular shapes among other shapes.

System 110 can identify and target a network of subject's brain 104. For example, system 110 can identify a network of subject's brain 104 to determine multiple target areas to stimulate that will stimulate a target area or produce a desired effect. Controller 112 of system 110 can then stimulate the multiple target areas sequentially or simultaneously to stimulate the target area.

In some implementations, controller 112 can control transducers 116 to stimulate multiple different target areas. For example, controller 112 can focus on or along two different points of a particular nerve using a two-dimensional phased array of transducers 116. In some implementations, controller 112 can control transducers 116 to target one area per array of transducers and/or per transducer. In some implementations, controller 112 controls transducers 116 to simultaneously stimulate two or more target areas. In some implementations, system 110 can stimulate multiple, smaller target areas within a single target area. For example, controller 112 can control transducers 116 to target multiple separate points along a single nerve for additional benefits. Controller 112 can focus multiple transducers 116 on a single target area. For example, controller 112 can control transducers 116 to sync pulses from multiple transducers to match, for example, a measured speed of a pain signal influx.

Controller 112 can control transducers 116 to provide multi-pulse superposition. A pulse at a single focal point makes a pressure wave that propagates radially outward. Controller 112 can use interference effects of ultrasonic emissions to stack a radially propagating pulse with a second pulse at a new position within a target. For example, controller 112 can produce ultrasonic beams in phase and at the same frequency to produce a constructive interference result. Controller 112 can move the transducers 116 to the new position or steer the transducers 116 to target the new position. Controller 112 can control the steering and focus of the superpositioned ultrasound pulses such that single-pulse thresholds for power are respected while building up displacement with pressure or shear waves from multiple pulses with different focal locations.

Controller 112 can use interference effects of ultrasonic emissions to generate an ultrasonic beat frequency. For example, controller 112 can generate multiple ultrasonic beams with different frequencies to create a beat frequency using both constructive and destructive interference effects. These beat frequencies (related to the differential between the original frequencies) can produce stronger effects than can be achieved using the multiple beams individually. The beat frequencies can, for example, increase spatial resolution and provide non-linear effects. High frequency emissions provide a higher level of precision (by increasing spatial resolution) and low frequency emissions offer a lower level of precision, but travel farther. Controller 112 can use interference effects of ultrasonic emissions, for example, to create a beat envelope that can penetrate the subject 102's skull or other bones around an emission having a frequency that otherwise would not penetrate the subject 102's skull.

Controller 112 can locally stimulate a target area to produce immediate effects, whereas stimulating a particular area such that the energy transmitted to the area is propagated to a target area can take a longer period of time.

System 110 stimulates subject's brain 104 using ultrasonic stimulation provided by the transducers 116. In some implementations, system 110 can stimulate subject's brain 104 using additional modalities such as electrical or magnetic stimulation. The configuration of system 110's transducers 116 are dependent on the modality of stimulation. For example, in some implementations in which system 110 uses magnetic stimulation techniques, transducers 116 can be located somewhere other than in close proximity to subject 102's head.

System 110 allows contemporaneous or near-contemporaneous detection and stimulation, facilitating a transcranial stimulation system that is able to target large-scale brain networks of subject's brain 104 in real-time and make adjustments to the stimulation based on the detected data. Detection and stimulation may alternate with a period of seconds or less to enable the real-time or near-real-time system. Detection and stimulation signals can be multiplexed. System 110 can also measure phase locking between large-scale brain networks, such that system 110 can apply stimulation to a target area of subject's brain 104 with a known phase delay from a reference signal. For example, controller 112 can apply stimulation, through electrical fields, to a target area of subject's brain 104 in-phase with contemporaneous or near-contemporaneous brain signal measurements.

Sensors 114 detect activity of subject's brain 104. Detection can be done using electrical, optical, and/or magnetic techniques, such as EEG, MEG, PET, and MRI, among other types of detection techniques. For example, sensors 114 can include non-invasive sensors such as EEG sensors, MEG sensors, among other types of sensors. In this particular implementation, sensors 114 are EEG sensors. Sensors 114 can include temperature sensors, infrared sensors, light sensors, heart rate sensors, and blood pressure monitors, among other types of sensors. In addition to detecting activity of the subject's brain 104, sensors 114 can collect and/or record the activity data and provide the activity data to controller 112. In some implementations, sensors 114 can perform sonic-based imaging such as acoustic radiation force-based elasticity imaging.

Sensors 114 can perform optical detection such that detection does not interfere with the frequencies generated by transducers 116. For example, sensors 114 can perform near-infrared spectroscopy (NIRS) or ballistic optical imaging through techniques such as coherence gated imaging, collimation, wavefront propagation, and polarization to determine time of flight of particular photons. Additionally, sensors 114 can collect biometric data associated with subject 102. For example, sensors 114 can detect the heart rate, eye movement, and respiratory rate, among other biometric data of the subject 102.

Sensors 114 provide the collected brain activity data and other data associated with subject 102 to controller 112.

Transducers 116 generate one or more electric fields at a target area within a subject's brain 104. System 110 includes multiple transducers 116, which can generate multiple fields that create an interfering region at a focal point, such as a target area within subject's brain 104. Transducers 116 can be, for example, electrodes. Transducers 116 can be powered by direct current or alternating current. Transducers 116 can be identical to each other. In some implementations, transducers 116 can include transducers made of different materials.

In some implementations, sensors 114 can include transducers that emit and detect electrical activity within the subject's brain 104. For example, transducers 116 can include one or more of sensors 114. In some implementations, transducers 116 include each of sensors 114; the same set of transducers can perform the stimulation and detection of brain activity in response to the stimulation. In some implementations, one subset of transducers may be dedicated to stimulation and another subset dedicated to detection. In some implementations, the stimulation system, i.e., transducers 116, and the detection system, i.e., sensors 114, are electromagnetically or physically shielded and/or separated from each other such that fields from one system do not interfere with fields from the other system. In some implementations, system 110 allows for contemporaneous or near-contemporaneous stimulation and measurement through, for example, the use of high performance filters that allow for high frequency stimulation at a high amplitude during low noise detection.

System 110 provides different effects depending on the spatial precision that can be achieved by transducers 116. For example, ultrasound emissions can provide higher spatial resolution than electrical or magnetic stimulation. System 110 can stimulate different nodes or portions of brain networks based on the resolution achievable by transducers 116. Controller 112 can target different sizes of spectral areas or different brain regions for different purposes.

Controller 112 includes one or more computer processors that control the operation of various components of system 110, including sensors 114 and transducers 116 and components external to system 110, including systems that are integrated with system 110. Controller 112 provides transcranial colored noise stimulation.

Controller 112 generates control signals for the system 110 locally. The one or more computer processors of controller 112 continually and automatically determine control signals for the system 110 without communicating with a remote processing system. For example, controller 112 can receive brain activity feedback data from sensors 114 in response to stimulation from transducers 116 and process the data to determine control signals and generate control signals for transducers 116 to alter or maintain one or more fields generated by transducers 116 within the target area of subject's brain 104.

Controller 112 controls sensors 114 to collect and/or record data associated with subject's brain 104. For example, sensors 114 can collect and/or record data associated with stimulation of subject's brain 104. In some implementations, controller 112 can control sensors 114 to detect the response of subject's brain 104 to stimulation generated by transducers 116. Sensors 114 can also measure brain activity and function through optical, electrical, and magnetic techniques, among other detection techniques.

Controller 112 is communicatively connected to sensors 114. In some implementations, controller 112 is connected to sensors 114 through communications buses with sealed conduits that protect against solid particles and liquid ingress. In some implementations, controller 112 transmits control signals to components of system 110 wirelessly through various wireless communications methods, such as RF, sonic transmission, electromagnetic induction, etc.

Controller 112 can receive feedback from sensors 114. Controller 112 can use the feedback from sensors 114 to adjust subsequent control signals to system 110. The feedback, or subject's brain 104's response to stimulation generated by transducers 116 can have frequencies on the order of tens of Hz and voltages on the order of pV. Subject's brain 104's response to stimulation generated by transducers 116 can be used to dynamically adjust the stimulation, creating a continuous, closed loop system that is customized for subject 102.

Controller 112 can be communicatively connected to sensors other than sensors 114, such as sensors external to the system 110, and uses the data collected by sensors external to the system 110 in addition to the sensors 114 to generate control signals for the system 110. For example, controller 112 can be communicatively connected to biometric sensors, such as heart rate sensors or eye movement sensors, that are external to the system 110.

Controller 112 can accept input other than EEG data from the sensors 114. The input can include sensor data from sensors separate from system 110, such as temperature sensors, light sensors, heart rate sensors, eye-tracking sensors, and blood pressure monitors, among other types of sensors. In some implementations, the input can include user input. In some implementations, and subject to safety restrictions, a subject can adjust the operation of the system 110 based on the subject's comfort level. For example, subject 102 can provide direct input to the controller 112 through a user interface. In some implementations, controller 112 receives sensor information regarding the condition of a subject. For example, sensors monitoring the heart rate, respiratory rate, temperature, blood pressure, etc., of a subject can provide this information to controller 112. Controller 112 can use this sensor data to automatically control system 110 to alter or maintain one or more fields generated within the target area of subject's brain 104.

In some implementations, controller 112 can monitor the subject's use of the system 110 to prevent overuse of the system. For example, controller 112 can monitor levels of use, such as the length of time that the system 110 is used or the strength of the settings at which the system 110 is used, to detect overuse or dependency and perform a safety function such as notifying the subject, stopping the system, or notifying another authorized user such as a healthcare provider. In one example, if the subject uses the system 110 for longer than a threshold period of time that is determined to be safe for the subject, the system 110 can lock itself and prevent further stimulation from being provided. In some implementations, the system 110 can enforce the threshold period of usage for the subject's safety over a period of time, such as 20 minutes of usage within 24 hours. In some implementations, the system 110 can enforce a waiting period between uses, such as remaining locked for 4 hours after a period of usage. Safety parameters such as the threshold period of usage, period of time, and waiting period, among other parameters, can be specified by the subject, the system 110's default settings, a separate system, and/or an authorized user such as a healthcare provider.

Controller 112 can use techniques such as facial recognition, skull shape recognition, among other techniques, for a subject's safety. For example, controller 112 can compare a detected skull shape of a current wearer of the system 110 to determine whether the wearer is an authorized subject. Controller 112 can also select particular models and settings based on the detected subject to personalize stimulation.

Controller 112 allows for input from a user, such as a healthcare provider or a subject, to guide the stimulation. Rather than being fixed to a specific random noise waveform, controller 112 allows a user to feed in waveforms to control the stimulation to a subject's brain.

Controller 112 uses data collected by sensors 114 and sources separate from system 110 to reconstruct characteristics of brain activity detected in response to stimulation from transducers 116, including the location, amplitude, frequency, and phase of large-scale brain activity. For example, controller 112 can use individual MRI brain structure maps to calculate electric field locations within a particular brain, such as subject's brain 104.

Controller 112 controls the selection of which of transducers 116 to activate for a particular stimulation pattern. Controller 112 controls the voltage, frequency, and phase of electric fields generated by transducers 116 to produce a particular stimulation pattern. In some implementations, controller 112 uses time multiplexing to create various stimulation patterns of electric fields using transducers 116. In some implementations, controller 112 turns on various combinations of transducers 116, which may have differing operational parameters (e.g., voltage, frequency, phase) to create various stimulation patterns of electric fields.

Controller 112 selects which of transducers 116 to activate and controls transducers 116 to generate fields in a target area of subject's brain 104 based on detection data from sensors 114 and stimulation parameters for subject 102. In some implementations, controller 112 selects particular transducers based on the position of the target area. For example, controller 112 can select opposing transducers closest to the target area within subject's brain 104. In some implementations, controller 112 selects particular transducers based on the stimulation to be applied to the target area. For example, controller 112 can select transducers capable of producing a particular voltage or frequency of electric field at the target area.

Controller 112 operates multiple transducers 116 to generate electric fields at the target area of subject's brain 104. Controller 112 operates multiple transducers 116 to generate electric fields using direct current or alternating current. Controller 112 can operate multiple transducers 116 to create interfering electric fields that interfere to produce fields of differing frequencies and voltage. For example, controller 112 can operate two opposing transducers 116 (e.g., transducers 116a and 116h) to generate two electric fields having frequencies on the order of kHz that interfere to produce an interfering electric field having a frequency on the order of Hz. Controller 112 can control operational parameters of transducers 116 to generate electric fields that interfere to create an interfering field having a particular beat frequency.

In some implementations, controller 112 can communicate with a remote server to receive new control signals. For example, controller 112 can transmit feedback from sensors 114 to the remote server, and the remote server can receive the feedback, process the data, and generate updated control signals for the system 110 and other components.

System 110 can receive input from subject 102 and automatically determine a target area and control transducers 116 to produce fields of particular voltage and frequency at the target area. For example, controller 112 can determine, based on collected feedback information from subject's brain 104 in response to stimulation, an area, or large-scale brain network, to target.

System 110 performs activity detection to uniquely tailor stimulation for a particular subject 102. In some implementations, the system 110 can start with a baseline map of brain conductivity and functionality and dynamically adjust stimulation to the target area of subject's brain 104 based on activity feedback detected by sensors 114. In some implementations, system 110 can perform tomography on subject's brain 104 to generate maps, such as maps of large-scale brain activity or electrical properties of the head or brain. For example, the system 110 can produce large-scale brain network maps for subject's brain 104 based on current absorption data measured by sensors 114 that indicate the amount of activity of a particular area of subject's brain 104 in response to a particular stimulus. In some implementations, system 110 can start with provisionally tailored maps that are generally applicable to a subset of subjects 102 having a set of characteristics in common and dynamically adjust stimulation to the target area of subject's brain 104 based on activity feedback detected by sensors 114.

In some implementations, controller 112 can control transducers 116 such that the current of the electric fields generated are lower than the current used in therapeutic applications. In some implementations, controller 112 can be used to produce electric field regions that affect the network state that a subject is in. For example, controller 112 can be used to produce interfering regions that induce a focused state, a relaxed state, or a meditation state, among other states, of subject's brain 104. In some implementations, controller 112 can be used to manipulate the state of subject's brain 104 to increase focus and/or creativity and aid in relaxation, among other network states.

Controller 112 can perform active, dynamic correction to the stimulation parameters, including the active correction for aberrations in the material through which the ultrasonic emissions will propagate. Such aberrations, such as variations in skull structure, hair, and other materials, can act as a barrier to the ultrasonic emissions and affect the actual impact of the ultrasonic stimulation on subject 102's brain tissue. For example, the skull structure can scatter and/or absorb ultrasonic emissions from system 110 and reduce the impact of the stimulation on subject's brain 104. Controller 112 can dynamically adjust the stimulation parameters to compensate, for example, for variation in skull structure from a baseline model based on sensor data from sensors 114 and data obtained from imaging ultrasonic emissions from transducers 116. In some implementations, controller 112 controls and utilizes lenses and other components to correct for structural aberrations. For example, controller 112 can operate focusing elements such as axicon-a special type of lens that has a conical surface and transforms beams into ring shaped distribution-Fresnel zone plates or Soret-an intense peak in the blue wavelength region of the visible spectrum-zone plates integrated with the transducers. Controller 112 can control elements such as the lenses and/or plates by moving, tilting, applying mechanical stress, applying electro-magnetic fields, and/or applying heat to the elements, among other techniques. In some implementations, each of the one or more transducers 116 includes a custom lens, delay line, or holographic beam former.

Controller 112 can adapt stimulation parameters based on subject 102's bone structure. For example, controller 112 can direct ultrasonic stimulation to different target areas of subject 102 based on the thickness of the bone at that area. In one example, controller 112 can direct stimulation through subject 102's temporal bone window, which is the thinnest part of the skull, in order to stimulate a target area of subject's brain 104 with the minimum amount of skull attenuation. Controller 112 can determine the thickness, shape, size, and/or location, among other characteristics, of particular skeletal structures of subject 102 and use the data to direct stimulation using the structures to aid or amplify the stimulation provided.

System 110 includes safety functions that allow a subject to use the system 110 without the supervision of a medical professional. In some implementations, system 110 can be used by a subject for non-clinical applications in settings other than under the supervision of a medical professional.

In some implementations, system 110 cannot be activated by a subject without the supervision of a medical professional, or cannot be activated by a subject at all. For example, system 110 may require credentials from a medical professional prior to use. In some implementations, only subject 102's doctor can turn on system 110 remotely or at their office.

In some implementations, system 110 can uniquely identify a subject 102, and may only be used by the subject 102. For example, system 110 can be locked to particular subjects and may not be turned on or activated by any other users.

System 110 can limit the range of frequencies and intensities of the stimulation applied through transducers 116 to prevent delivery of harmful patterns of stimulation. For example, system 110 can detect and classify stimulation patterns as seizure-inducing, and prevent delivery of seizure inducing stimulus. In some implementations, system 110 can detect activity patterns in early stages of the activity and preventatively take action. For example, system 110 can detect activity patterns in an early stage of anxiety and preventatively take action to prevent subject's brain 104 from progressing into later stages of anxiety. System 110 can also detect seizure activity patterns using the extracranial activity and biometric data collected by sensors 114, and adjust the stimulation provided by transducers 116 to prevent subject 102 from having a seizure.

In some implementations, system 110 is used for therapeutic purposes. For example, system 110 can be tailored to a subject 102 and used as a brain activity regulation device that detects epileptic activity within the subject's brain 104 and provides prophylactic stimulation.

Controller 112 can use statistical and/or machine learning models which accept sensor data collected by sensors 114 and/or other sensors as inputs. The machine learning models may use any of a variety of models such as decision trees, linear regression models, logistic regression models, neural networks, classifiers, support vector machines, inductive logic programming, ensembles of models (e.g., using techniques such as bagging, boosting, random forests, etc.), genetic algorithms, Bayesian networks, etc., and can be trained using a variety of approaches, such as deep learning, association rules, inductive logic, clustering, maximum entropy classification, learning classification, etc. In some examples, the machine learning models may use supervised learning. In some examples, the machine learning models use unsupervised learning.

Power system 150 provides power to the various subsystems of system 100 and is connected to each of the subsystems. Power system 150 can also generate power, for example, through renewable methods such as solar or mechanical charging, among other techniques.

In this particular example, power system 150 is shown to be separate from the various other subsystems of system 100. Power system 150 is, in this example, an external power source housed within a separate form factor, such as a waist pack connected to the various subsystems of system 100.

In some implementations, system 100 can be used without an external power source. For example, system 100 can include an integrated power source or an internal power source. The integrated power source can be rechargeable and/or replaceable. For example, system 100 can include a replaceable, rechargeable battery pack that provides power to the transducers and sensors and is housed within the same physical device as system 100.

In this particular example, system 100 is housed within a wearable headpiece that can be placed on a subject's head. In some implementations, system 100 can be implemented as a network of individual transducers and sensors that can be placed on the subject's head or a device that holds individual transducers and sensors in fixed positions around the subject's head. In some implementations, system 100 can be implemented as a device tethered in place and is not portable or wearable. For example, system 100 can be implemented as a device to be used in a specific location within a healthcare provider's office.

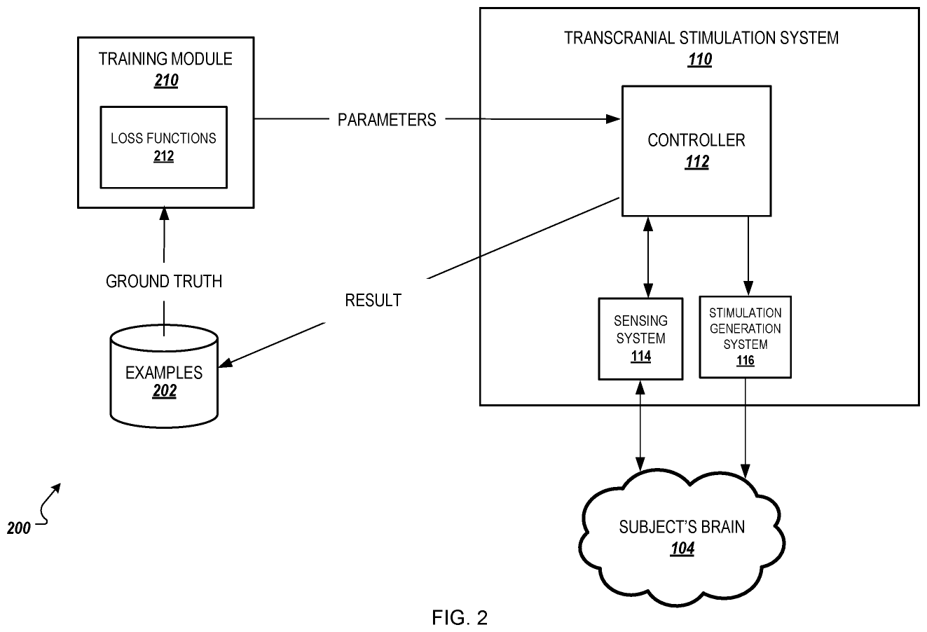

FIG. 2 is a diagram of an example block diagram of a system 200 for training a transcranial stimulation system. For example, system 200 can be used to train transcranial stimulation system 110 as described with respect to FIG. 1.

As described above with respect to FIG. 1, system 110 includes a controller 112 that classifies brain activity detected by a sensing system and determines stimulation parameters for a stimulation pattern generation system. For example, controller 112 classifies activity detected by sensors, or sensing system 114, and determines stimulation parameters for transducers, or stimulation pattern generation system 116, including the pattern, frequency, duty cycle, shape, power, and modality. Activity classification can include identifying the location, amplitude, entropy, frequency, and phase of large-scale brain activity. Controller 112 can additionally perform functions including quantifying dosages and effectiveness of applied stimulation.

Examples 202 are provided to training module 210 as input to train a machine learning model used by controller 112, such as an activity classification model. Examples 202 can be positive examples (i.e., examples of correctly determined activity classifications) or negative examples (i.e., examples of incorrectly determined activity classifications).

Examples 202 include the ground truth activity classification, or an activity classification defined as the correct classification. Examples 202 include sensor information such as baseline activity patterns or statistical parameters of activity patterns for a particular subject. For example, examples 202 can include tomography data of subject 102's brain 104 generated through activity detection performed by sensors 114 or sensors external to system 110 as described above (e.g., MRIs, EEGs, MEGs, and computed tomography based on the detected data from sensors 114, among other detection techniques). Examples 202 can include statistical parameters of noise patterns of subject 102's brain 104.

In some implementations, the statistical parameters of subject 102's brain 104's noise patterns are closely related to entropic measurements of the patterns. The entropic measurements and noise patterns can be overlapping and capture many of the same properties for the purposes of analyzing the noise patterns.

The ground truth indicates the actual, correct classification of the activity. For example, a ground truth activity classification can be generated and provided to training module 210 as an example 202 by detecting an activity, classifying the activity, and confirming that the activity classification is correct. In some implementations, a human can manually verify the activity classification. The activity classification can be automatically detected and labelled by pulling data from a data storage medium that contains verified activity classifications.

The ground truth activity classification can be correlated with particular inputs of examples 202 such that the inputs are labelled with the ground truth activity classification. With ground truth labels, training module 210 can use examples 202 and the labels to verify model outputs of an activity classifier and continue to train the classifier to improve forward modelling of brain activity through the use of detection data from sensors 114 to predict brain functionality and activity in response to stimulation input.

The sensor information guides the training module 210 to train the classifier to create a morphology correlated map. The training module 210 can associate the morphology of a particular subject's brain 104 with an activity classification to map out brain conductivity and functionality. Inverse modelling of brain activity can be conducted by using measured responses to approximate brain networks that could produce the measured responses. The training module 210 can train the classifier to learn how to map multiple raw sensor inputs to their location within subject's brain 104 (e.g., a location relative to a reference point within subject's brain 104's specific morphology) and activity classification based on a morphology correlated map. Thus, the classifier would not need additional prior knowledge during the testing phase because the classifier is able to map sensor inputs to respective areas within subject's brain 104 and classify activities using the correlated map.

Training module 210 trains an activity classifier to perform activity classification. For example, training module 110 can train a model used by controller 112 to recognize large-scale brain activity based on inputs from sensors within an area of subject's brain 104. Training module 210 refines controller 112's activity classification model using electrical tomography data collected by sensors 114 for a particular subject's brain 104. Training module 210 allows controller 112 to output complex results, such as a detected brain functionality instead of, or in addition to, simple imaging results.

Controller 112 can, for example, adjust brain stimulation patterns based on detected activity patterns. For example, controller 112 may adjust stimulation parameters and patterns based on, for example, a property of brains and brain signals known as criticality, where brains can flexibly adapt to changing situations.

In some implementations, controller 112 can apply stimulation patterns that amplify natural brain activity. For example, controller 112 can detect and identify natural activity patterns of brain signals. In one example, an identified activity pattern includes pink noise pattern. Activity patterns can vary, for example, in frequency, power, and/or wavelength.

System 110 performs monitoring of the effects of stimulation. The monitoring can be performed using various methods of measurement. In some implementations, controller 112 can detect and classify psychological states of a subject's brain 104 based on physiological input data. For example, controller 112 can receive input data including eye movements and other biometric measurements. Controller 112 can use eye movement data, for example, to detect cognitive load parameters.

In some implementations, controller 112 can correlate physiological signals with a subject's brain state. For example, controller 112 can calculate an entropic state of subject 102's brain state based on subject 102's eye movement.

In some implementations, system 110 can be a closed-feedback user-guided stimulation system, that is driven by user feedback such that stimulation at a particular time is a function of feedback from previous times. For example, feedback can include user feedback provided through a user interface, such as pushing one button when the effect of stimulation is trending in a positive direction and is achieving a desired effect and pushing a different button when the effect of stimulation is trending in a negative direction and is achieving an undesired effect, among other techniques and modalities of feedback systems.

System 110 can receive feedback directly from subject 102 in addition to the biofeedback (e.g., biological signals such as heart rate, oxygen levels, etc.) detected by sensors 114. For example, system 110 can receive auditory or visual guidance from subject 102. In some implementations, controller 112 can receive visual guidance from subject 102. For example, subject 102 can provide visual guidance to system 110 through a photodetector or camera sensor 114 by making a gesture or other visual signal.

In some implementations, controller 112 can receive, for example, verbal output from a subject 102. For example, controller 112 can use techniques such as natural language processing to classify a subject 102's statements. These classifications can be used to determine whether a subject is in a particular psychological state. The system can then use these classifications as feedback to determine stimulation parameters to adjust the stimulation provided to the subject's brain. For example, controller 112 can determine, based on verbal feedback, the emotional content of subject 102's voice and subject 102's brain state. Controller 112 can then determine stimulation parameters to adjust the stimulation provided to subject 102's brain in order to guide subject 102 to a different state or amplify subject 102's current state. For example, controller 112 can perform task-based feedback and classification, where a subject 102 is asked to perform tasks during the stimulation, and subject 102's performance of the task or verbal feedback during their performance of the task is used to determine the subject 102's brain state.

In some implementations, controller 112 can tailor stimulation based on a measure of the subject's attention or direct subjective feedback, such as how the stimulation makes a subject feel. Feedback can also be derived from the monitoring of peripheral physiological signals, such as, but not limited to, heart rate, heart rate variability, pupil dilation, blink rate, and related measures. In some implementations, controller 112 can monitor, for example, the amount and composition of a subject's sweat to be used as an indication of sympathetic nervous system engagement. These, and other biomarkers can be used alone or in combination to model the state of the subject's brain activity and/or peripheral nervous system and adjust stimulation parameters accordingly, or even, as a way to quantify the effective dosage of stimulation. For example, stimulation of the cranial nerve (i.e., vagus nerve stimulation) can be quantified by measuring the dilation of a subject's pupil.

In some implementations, system 110 can provide auditory or visual guidance to the subject 102. For example, system 110 can guide the user through a meditation or relaxation routine that allows the user to assist in improving the effects of the transcranial stimulation performed by system 110.

Training module 210 trains controller 112 using one or more loss functions 212. Training module 110 uses an activity classification loss function 212 to train controller to classify a particular large-scale brain activity. Activity classification loss function 212 can account for variables such as a predicted location, a predicted amplitude, a predicted frequency, and/or a predicted phase of a detected activity.

Training module 210 can train controller 112 manually or the process could be automated. For example, if an existing tomographic representation of subject's brain 104 is available, the system can receive sensor data indicating brain activity in response to a known stimulation pattern to identify the ground truth area within subject's brain 104 at which an activity occurs through automated techniques such as image recognition or identifying tagged locations within the representation. A human can also manually verify the identified areas.

Training module 210 uses the loss function 112 and examples 202 labelled with the ground truth activity classification to train controller 112 to learn where and what is important for the model. Training module 210 allows controller 112 to learn by changing the weights applied to different variables to emphasize or deemphasize the importance of the variable within the model. By changing the weights applied to variables within the model, training module 210 allows the model to learn which types of information (e.g., which sensor inputs, what locations, etc.) should be more heavily weighted to produce a more accurate activity classifier.

Training module 210 uses machine learning techniques to train controller 112, and can include, for example, a neural network that utilizes activity classification loss function 212 to produce parameters used in the activity classifier model. These parameters can be classification parameters that define particular values of a model used by controller 112.

In some implementations, a model used by controller 112 can select a filter to apply to the generated stimulation pattern to stabilize the stimulation being applied to subject 102 when subject 102's brain activity reaches a particular level of complexity.

Controller 112 classifies brain activity based on data collected by sensors 114. Controller 112 performs forward modelling of brain activity and inverse modelling of brain activity, given base, reasonable assumptions regarding the stimulation applied to a target area within subject's brain 104.

Forward modelling allows controller 112 to determine how to propagate waves through subject's brain 104. For example, controller 112 can receive a specified objective (e.g., a network state of subject's brain 104) and design stimulation field patterns to modify brain activity detected by sensors 114. Controller 112 can then control two or more transducers 116 to apply electrical fields to a target area of subject's brain 104 to produce the specified objective network state.

Inverse modelling allows controller 112 to estimate the most likely relationship between the detected activity and the corresponding areas or networks of subject's brain 104. For example, controller 112 can receive brain activity data from sensors 114 and reconstruct, using an activity classifier model, the location, amplitude, frequency, and phase of the large-scale brain activity. Controller 112 can then dynamically alter the existing activity classifier model and/or tomography representation of subject's brain 104 based on the reconstruction.

Controller 112 can access, create, edit, store, and delete models that are tailored to particular common skull structures and/or brain structures. Controller 112 can use different combinations of models for skull structure and brain network structure. Each of these models can be further customized for a subject 102. Controller 112 has access to a set of models that are individualized to a certain extent. For example, controller 112 can use general models for people having a large skull, a small skull, a more circular skull, a more oblong skull, etc. These models provide a starting point that is closer to a subject's skull and brain structures than a single model.

Controller 112 can alter models and create more granularity in the models or otherwise define general models that are often used to be stored within a storage medium available to system 110. Controller 112 can maintain a single model for a particular subject 102 that is improved over time for the subject 102.

The models allow controller 112 to individualize stimulation and treatment to each subject, by using machine learning to select and adjust stimulation parameters for a subject's individual anatomy and brain and/or skull structure. For example, the models allow controller 112 to maximize the impact of the ultrasonic stimulation on brain tissue and other target areas by adjusting for a subject's skull structure and the location of particular regions of subject's brain 104.

In some implementations, controller 112 can use structural features of subject 102's head. For example, controller 112 can use features such as the location and structure of a subject 102's jaw, cheekbone, and nasal bridge to calibrate a model and adjust stimulation for the subject 102. In some implementations, controller 112 can limit the features to those local to the target area for stimulation. Controller 112 can, for example, use a 3D reconstruction of subject 102 based on photos or video taken of subject 102. In some implementations, controller 112 can use other imaging data such as acoustic-based imaging, electrical, and/or magnetic imaging techniques.

In some implementations, controller 112 can use external structural features to calibrate a model and to adjust stimulation targeting and parameters. For example, system 110 can be integrated with a helmet structure that includes a fluid-filled sac or other adjustable, flexible structure that ensures a tight fit on subject 102's head. In some implementations, system 110 can be integrated with a helmet structure that includes an inflatable structure that can be adjusted to exert more or less pressure on subject 102's head to adjust the fit of the helmet.

System 110 can be implemented with a physical form factor that can correct for any aberrations or variations in subject 102's skull structure or other physical features from a general model. For example, system 110 can be implemented as a helmet with a personalized three-dimensional insert. The personalized insert can correct for subject 102's particular variations in skull structure, for example, from a general model of an oval-shaped skull to allow close contact with target portions of subject 102's skull. The personalized insert can be made from material selected for its conductive properties, its texture, etc. In some implementations, controller 112 can control the shape and size of the insert. In some implementations, the insert is fabricated with a fixed shape and can be changed for each subject 102.

In some implementations, the personalized insert can be shaped to provide an improved surface along which transducers are placed and/or through which ultrasonic stimulation is performed. For example, the personalized insert can be shaped to provide a uniform, hemispherical transducer surface. In some implementations, the personalized insert can be shaped to allow all stimulation to arrive at a target area at the same time. The personalized insert can be shaped to provide a reflective surface for the ultrasonic stimulation to direct and/or focus the stimulation. For example, the personalized insert can be shaped to focus the stimulation at a particular target area.

In some implementations, the personalized insert can be shaped to provide a non-uniform surface that is thicker in some areas than in other areas. For example, the personalized insert can be shaped to create a delay line in propagation along a target area. The personalized insert can be shaped based on a calculation of skull thickness performed using imaging techniques as described above or other sensor data collected and provided to controller 112.

In some implementations, the personalized insert can be shaped to create time and/or phase delays in the ultrasonic stimulation. For example, the personalized insert can be shaped to create a phase-delay in ultrasound beams transmitted through the insert based on properties of the material of the insert, including the refractive index, the thickness, and the shape, among other properties. The personalized insert can be designed to correct for anomalous structures and cavities in certain regions of the subject 102's skull by redirecting emissions.

The structure of the personalized insert can be based, for example, on imaging data from, a scan of subject 102's skull that produces a three-dimensional representation of the external structure of the subject 102's skull. For example, the structure of the personalized insert can be determined based on an ultrasound, an MRI, a CT scan or an image of subject 102's skull structure generated from other imaging techniques. In some implementations, the structure of the personalized insert can be based on a general structure of a typical human skull model and adjustments can be made based on imaging data.

An initial structure of the personalized insert can be individualized to a certain extent. For example, controller 112 can use general models for people having a particular type of skull aberration, people having typical skull shapes, etc. These models provide a starting point that is closer to a subject's skull and brain structures than a single insert for a general skull size.

Controller 112 can use various types of models, including general models that can be used for all patients and customized models that can be used for particular subsets of patients sharing a set of characteristics, and can dynamically adjust the models based on detected brain activity. For example, the classifier can use a base network for subjects and then tailor the model to each subject.

Controller 112 can detect and classify brain activity using sensors 114 contemporaneously or near-contemporaneously with the stimulation provided by transducers 116. In some implementations, the brain activity can be detected through techniques performed by systems external to system 110, such as functional magnetic resonance imaging (fMRI) or diffusion tensor imaging (DTI).

In some implementations, controller 112 provides stimulation that matches patterns of the natural signals of a subject's brains. Humans shift across brain activity patterns similar to patterns of noise. For example, human brain activity patterns can shift from Brownian noise patterns having low frequencies during sleep, to pink noise patterns as a subject wakes up, to pink and/or white noise patterns as a subject becomes more active. Controller 112 can detect and identify brain activity patterns of a subject 102 and determine, for example, statistical parameters of random noise stimulation patterns that match subject 102's naturally occurring brain activity patterns to amplify the effects of the stimulation. Matching subject 102's naturally occurring brain activity patterns can produce better phase alignment.

Controller 112 can determine, for example, stimulation patterns that match subject 102's naturally occurring Brownian noise patterns, pink noise patterns, and white noise patterns. Controller 112 can then apply white noise patterns to subject 102's brain 104 when subject 102 should be in an active brain state. For example, controller 112 can aid in focus and alertness by matching its patterns of stimulation to subject 102's brain 104's naturally occurring white noise pattern to amplify the effects of stimulation.

In some implementations, controller 112 can apply a signal to the subject's brain to sync the brain to a particular pattern and then transition to a different stimulation pattern. By matching subject 102's brain 104's naturally occurring activity pattern, controller 112 can, in effect, grab the attention of brain 104. Controller 112 can then transition to a different stimulation pattern, leading brain 104 to a different activity pattern.

As described above, system 110 can include MEG, EEG, and/or MRI imaging sensors. Controller 112 can use the imaging data from sensors 114 to adjust stimulation. In some implementations, controller 112 can use transducers of the stimulation generation system 116 to perform imaging functions. For example, controller 112 can control transducers 116 to operate at imaging frequencies and using imaging level parameters to perform ultrasound imaging. Controller 112 can, for example, perform tissue displacement ultrasound imaging to confirm that the stimulation generated by stimulation generation system 116 is being directed to the correct target area within the subject's brain 104. The imaging performed by controller 112 may be performed using the same transducers 116 that perform the stimulation, and in some implementations, the image quality may not be as detailed or clear as clinical quality imaging, but can be used by controller 112 to dynamically adjust stimulation parameters and/or steer and direct stimulation.

In addition to matching the statistical activity patterns, controller 112 can also measure the power spectral density of a subject 102's brain state and reproduce the patterns to assist brain 104 in matching the stimulation. For example, controller 112 may want to limit the amount of power provided in the applied stimulation, but the stimulation needs to be of enough power to produce a response. By matching the power spectral density of a brain 104's state, controller 112 can induce maximum self-organized complexity such that brain 104 is guided by later changes in stimulation.

Controller 112 can determine the complexity of a noise pattern occurring in a subject's brain using several different methods of measurement. In some implementations, the complexity of brain signals matches the complexity of the subjective experience a subject is undergoing. For example, brain signals may have limited complexity when a subject is in deep sleep, whereas brain signals may have more complexity when a subject is under the influence of a stimulant.

Controller 112 provides a user with the ability to apply waveforms with various parameters as stimulation to a subject's brain. In some implementations, a user can select a particularly shaped waveform to apply to subject 102's brain 104. For example, a user can apply a triangle wave stimulation pattern to subject 102's brain 104. Different shapes of waveforms can have different effects. Applying a triangle wave stimulation pattern to a subject 102's brain 104 can act as a siren, seizing the attention of brain 104. A user can apply different shapes of wave stimulation patterns including sawtooth, sine, and square waves, among other shapes, to achieve different effects.

Controller 112 can collect response data from subject 102 to quantify dosage provided to subject 102's brain 104. For example, controller 112 can use trained models to quantify dosage based on a response from subject 102's brain 104 to stimulation. System 110 can implement limits on the amount of time that the system 110 can be used, monitor the cumulative dose delivered to various brain areas, enforce a maximum amount of current that can be output by transducers 116, or administer integrated dose control.

There has previously been no way to quantify the dosage of vagus nerve stimulation. Controller 112 provides a method of dosage quantification by measuring, for example, physiological responses, such as pupil dilation, to stimulation according to a particular set of parameters. Controller 112 can continuously track eye movement, pupil dilation, and other physiological responses and quantify how effective a particular set of stimulation parameters is.

In some implementations, controller 112 can quantify the effectiveness of a particular set of stimulation parameters by monitoring a differential response. For example, controller 112 can effectively “trap and trace” brain signals, such as pain signals, originating from a subject's brain. By comparing the characteristics of the brain signals, controller 112 can detect differential changes in response from a subject 102.

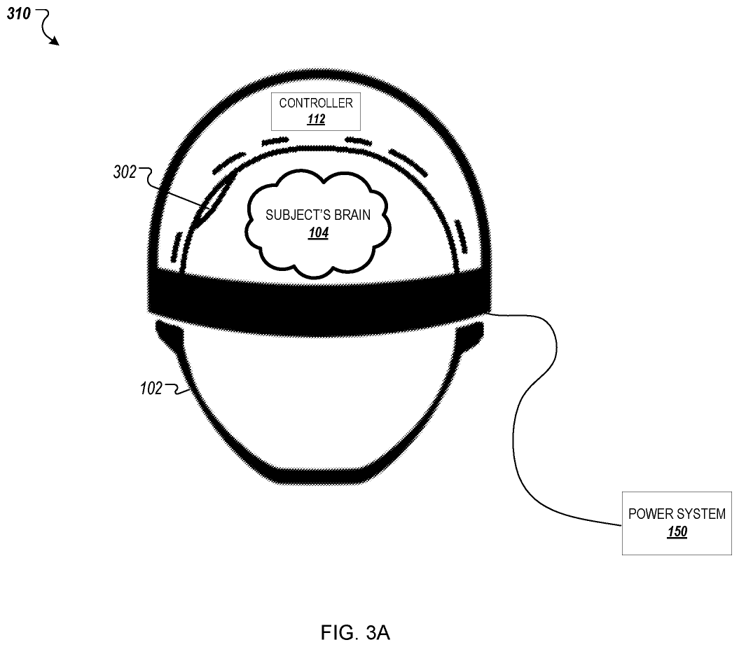

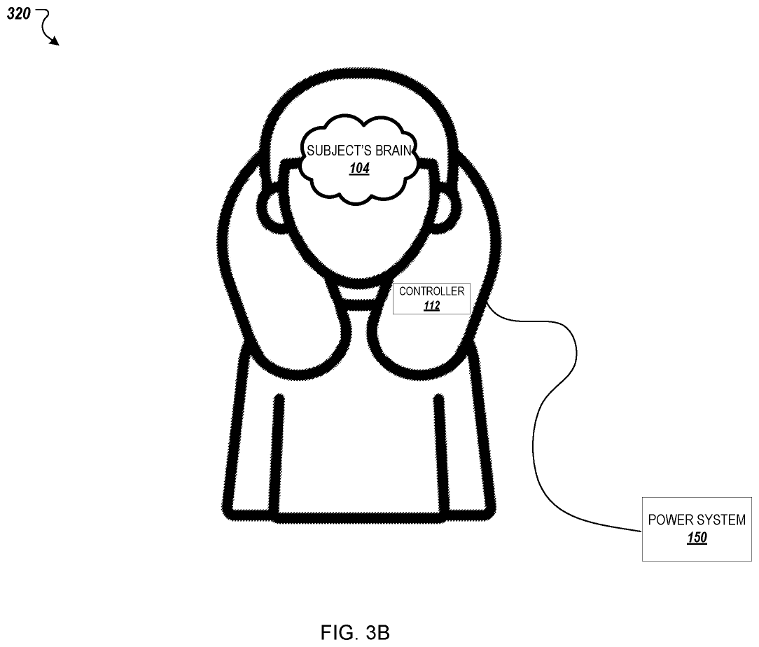

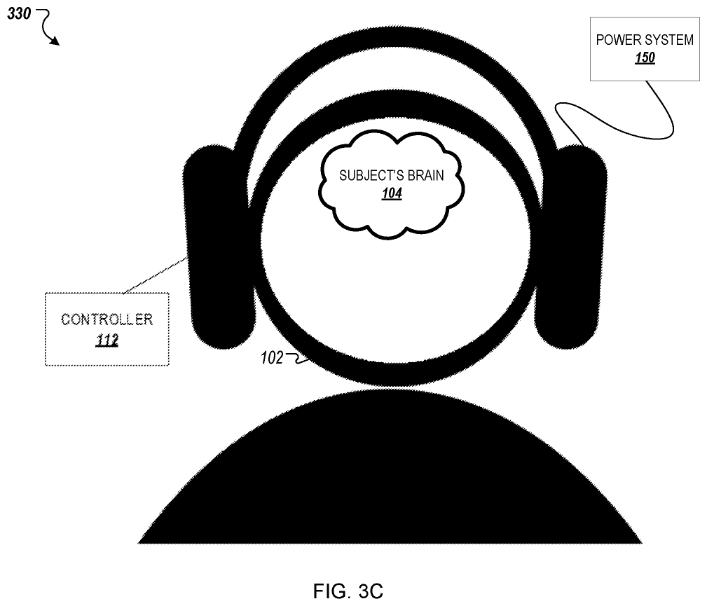

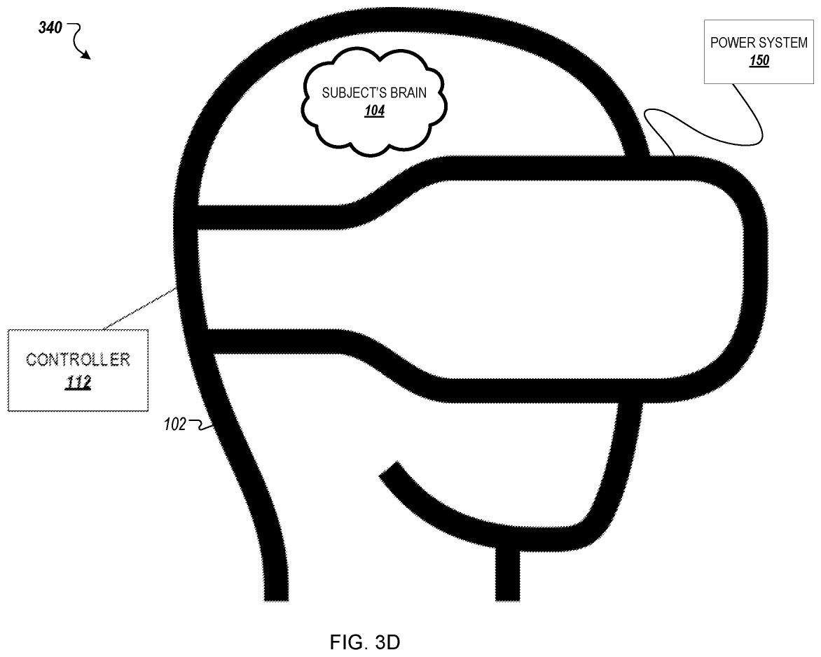

FIGS. 3A and 3B illustrate example form factors of a transcranial stimulation system that delivers transcranial stimulation to a target within a subject's brain. Other form factors for the transcranial stimulation system described in the present application are contemplated. For example, system 110 as described above with respect to FIGS. 1-2 can include devices such as devices 300 and 350 that each includes sensors 114 and/or transducers 116.

The devices illustrated in FIGS. 3A and 3B can be administered by a healthcare provider to a patient. In some implementations, the devices illustrated in FIGS. 3A and 3B can be operated by subject 102 without the supervision of a healthcare provider. For example, devices 300 and 350 can be provided to patients and can be adjustable by the patient, and in some implementations, can automatically calibrate to the patient and one or more particular target areas within subject's brain 104. The dynamic stimulation process is described above with respect to FIGS. 1-2.

While controller 112 is depicted as separate from the devices 300 and 350, controller 112 and associated power systems can be integrated with the devices of FIGS. 3A and 3B to provide a comfortable, more compact form factor. In some implementations, controller 112 communicates with a remote computing device, such as a server, that trains and updates controller 112's machine learning models. For example, controller 112 can be communicatively connected to a cloud-based computing system.

As described above, system 110 can include safety features to protect subject 102 and ensure the safe use of system 110. For example, system 110 can include a safety lock-out feature that prevents the transducers 116 from emitting pulses or beamforming if subject 102's head or other body part is not in a correct, safe position relative to the system 110.

FIGS. 3A, 3B, 3C, and 3D illustrate example form factors of a transcranial stimulation system that delivers transcranial stimulation to a target within a subject's brain. Other form factors for the transcranial stimulation system described in the present application are contemplated. For example, system 110 as described above with respect to FIGS. 1-2 can include devices such as devices 310, 320, 330, and 340 that each includes sensors 114 and/or transducers 116.

The devices illustrated in FIGS. 3A, 3B, 3C, and 3D can be administered by a healthcare provider to a patient. In some implementations, the devices illustrated in FIGS. 3A, 3B, 3C, and 3D can be operated by subject 102 without the supervision of a healthcare provider. For example, devices 310, 320, 330, and 340 can be provided to patients and can be adjustable by the patient, and in some implementations, can automatically calibrate to the patient and a particular target spot. Automatic targeting and calibration are described above with respect to FIG. 2.

While controller 112 is depicted as separate from the devices 310, 320, 330, and 340, controller 112 and associated power systems, such as power system 150, can be integrated with the devices of FIGS. 3A, 3B, 3C, and 3D to provide a comfortable, more compact form factor. In some implementations, controller 112 communicates with a remote computing device, such as a server, that trains and updates controller 112's machine learning models. For example, controller 112 can be communicatively connected to a cloud-based computing system.

FIG. 3A illustrates a device 310 that can be worn by a subject 102 on their head. In this particular implementation, device 310 is in a comfortable form factor that contacts subject 102 on multiple points on their head and has the transcranial stimulation system 110 as described in FIGS. 1-2. For example, device 310 can be a helmet.

System 110 can be implemented in a flexible, wearable form factor. For example, system 110 can use flexible transducers that allow the physical form factor of the system 110 to be portable, wearable, and adaptable to a subject 102.

For example, the system 110 can be implemented as a wireless helmet that contacts subject 102 on two or more points of their head. In some implementations, the system 110 can be a cap or headphones. In some implementations, the system 110 can be integrated into a headset that includes visual or auditory stimulation.

The device 310 that houses system 110 can include an insert 302 tailored to the shape of subject 102's skull to improve contact and/or coupling with subject 102's skull. For example, system 110's array of transducers 116 can be arranged according to the shape of the insert or the form factor of the system 110. The insert 302 can be, for example, a personalized insert as described above with respect to FIG. 2. The insert 302 can be a part of a coupling system of the transcranial ultrasonic stimulation system 110. The coupling system can improve the coupling between the transducers and the subject. In some implementations, the coupling system includes a cooling system that includes cooling fluid.

FIG. 3B illustrates a device 320 that can be worn by a subject 102 around their head and neck. In this particular implementation, device 320 is in a comfortable form factor in the shape of a pillow that is filled with fluid and has the stimulation generation and dynamic adjustment system as described in FIGS. 1-2. The pillow can either be filled with cooling fluid or made of material having a high thermal mass that allows for heat dissipation. The fluid-filled pillow provides a low-loss medium through which ultrasonic stimulation can be provided. Additionally, the fluid-filled pillow can be conformal to the subject 102's head and/or body to provide a better contact surface for the ultrasonic stimulation. The pillow can provide active cooling for the transcranial stimulation system 110. In some implementations, the system 110 includes a separate heat sink. In some implementations, the fluid-filled pillow can be a part of a coupling system of the transcranial ultrasonic stimulation system 110 that improves the coupling between the transducers and the subject.

In some implementations, the pillow is designed to support subject 102's head and neck. In some implementations, the pillow is designed to support other portions of subject 102's body. The fluid can be selected to improve contact and/or coupling of the system 110 and its transducer 116 to subject 102's body. In some implementations, the fluid can be selected to improve cooling of system 110 and reduce heat produced by the system 110's stimulation of subject 102.

The fluid can also be used to adjust beam placement and depth, among other parameters, to adjust the stimulation provided to subject 102. For example, the amount and composition of fluid within the pillow can be adjusted to change the characteristics and focal area, among other parameters, of one or more lenses placed between transducers 116 and a target within subject's brain 104. In some implementations, the fluid within the pillow can be manipulated to adjust the focal depth of the beam of ultrasonic stimulation to a target area. For example, given a known focal depth, the controller 112 can inflate and/or deflate the fluid-filled pillow by increasing or decreasing the amount of fluid, ratio of substances within the fluid, or the amount of air within the fluid-filled pillow in order to adjust the focal depth for the stimulation directed through the fluid-filled pillow.

In some implementations, the fluid within the pillow can be a material having propagation properties (such as a refractive index, density, etc.) having a correlation with electromagnetic fields. For example, the fluid within the pillow can have propagation properties correlated with electric fields. and system 110 can perform electric-field actuated adjustments of the properties of the fluid by emitting electric fields. In one example, the fluid can be on a surface with a pattern of transducers, and controller 112 can alter the properties of the fluid to change material properties of the fluid. In some implementations, the material properties of the fluid can be pressure or mechanically influenced. For example, controller 112 can alter the material properties of the fluid by applying mechanical stress to the fluid by increasing the pressure within a volume in which the fluid is contained.

The system 110 can be integrated into other items, such as pieces of furniture or components of vehicles or other applications. For example, the system 110, in pillow form, can be integrated into the headrest of a reclining chair or massage chair to aid in relaxation, or the headrest of a car to improve focus. The system 110 can be integrated into other vehicles, including airplanes and trains, among other vehicles and applications. For example, the system 110 can be integrated into the headrest of an airplane passenger seat to reduce flight-related anxiety or motion sickness, into a pilot or long-haul truck driver's seat to improve focus, and/or in a clinical setting to aid in therapy or other treatment, such as an MRI machine headrest to help with claustrophobia when being scanned, among other applications.

FIG. 3C illustrates a device 330 that can be worn by a subject 102 on their head. In this particular implementation, device 330 is in a comfortable form factor that contacts subject 102 on either side of their head and has the automatic steering and focusing systems as described in FIGS. 1-2. For example, device 330 can be a pair of headphones.

FIG. 3D illustrates a device 340 that can be worn by a subject 102 on their face. In this particular implementation, device 340 is in a comfortable form factor in the shape of eyewear and has the automatic steering and focusing systems as described in FIGS. 1-2. For example, device 340 can be a pair of glasses or goggles.

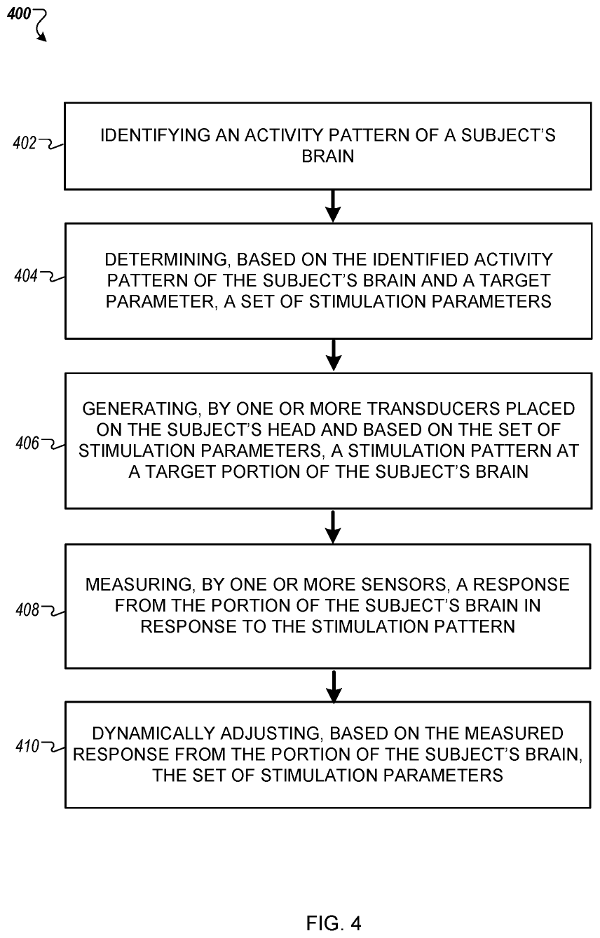

FIG. 4 is a flow chart of an example process 400 of transcranial stimulation of large-scale brain networks. Process 400 can be implemented by transcranial stimulation systems such as system 110 as described above with respect to FIGS. 1, 2, 3A, 3B, 3C, and 3D. In this particular example, process 400 is described with respect to system 110 in the form of a portable headset or helmet that can be used by a subject without the supervision of a medical professional. Briefly, according to an example, the process 400 begins with identifying an activity pattern of a subject's brain (402). For example, controller 112 can measure and identify an activity pattern of subject 102's brain 104.

The process 400 continues with determining, based on the identified activity pattern of the subject's brain and a target parameter, a set of stimulation parameters (404). For example, controller 112 can determine, based on identifying that subject 102's brain 104 is in a stress activity pattern and a target of a calm activity pattern, a set of stimulation parameters. The target parameter can include, for example, a target brain state, a target activity pattern, a user input of a particular waveform, an power of stimulation, a target object, a target size, a target composition, a duration of stimulation, a particular dosage of stimulation, a target quantification of reduction in pain, and/or a target percentage in reduction of tremors, among other parameters. The stimulation parameters can include, for example, a power, a waveform, a shape, a pattern, a statistical parameter, a duration, a modality (e.g., ultrasound, electrical, and/or magnetic stimulation, among other modes), a frequency, a period, a target location, a target size, and/or a target composition, among other parameters.

The process 400 continues with generating, by one or more ultrasound transducers placed on a subject's head and based on the set of stimulation parameters, a stimulation pattern at a portion of the subject's brain (406). For example, controller 112 can operate two transducers, 116a and 116f, to generate a calming stimulation pattern based on the set of stimulation parameters at a target area within the subject 102's brain 104.

The process 400 continues with measuring, by one or more sensors, a response from the portion of the subject's brain in response to the stimulation pattern (408). For example, controller 112 can operate sensors 114 to measure, within a few seconds, and thus contemporaneously or near-contemporaneously with the generating step, brain activity from the target area within the subject's brain 104. For example, sensors 114 can detect, using EEG, brain activity from the target area within the subject's brain 104 in response to the white noise stimulation pattern.

The process 400 concludes with dynamically adjusting, based on the measured response form the portion of the subject's brain, the set of stimulation parameters (410). For example, controller 112 can determine, based on the measured brain activity detected by sensors 114, that subject 102 is slowly entering a relaxed brain or network state, but has not reached the target calm activity pattern. Controller 112 can then determine, using the measured brain activity and the target calm activity pattern, stimulation parameters for transducers 116 to continue inducing the calm network state in the subject's brain 104. Controller 112 can operate transducers 116 according to the determined stimulation parameters to adjust the stimulation pattern. For example, controller 112 can operate transducers 116 to alter the frequency and amplitude of the stimulation pattern, thus facilitating a closed loop transcranial stimulation system for large-scale brain networks. Controller 112 can operate transducers 116 with a phase shift relative to a detected in-phase large-scale brain network, enhancing or decreasing the phase lock of the large-scale brain network. Controller 112 can operate transducers 116 with a frequency shift relative to a detected in-phase large-scale brain network, increasing or decreasing the frequency of the phase-locked large-scale brain network.

A number of implementations have been described. Nevertheless, it will be understood that various modifications may be made without departing from the spirit and scope of the disclosure. For example, various forms of the flows shown above may be used, with steps re-ordered, added, or removed.

All of the functional operations described in this specification may be implemented in digital electronic circuitry, or in computer software, firmware, or hardware, including the structures disclosed in this specification and their structural equivalents, or in combinations of one or more of them. The techniques disclosed may be implemented as one or more computer program products, i.e., one or more modules of computer program instructions encoded on a computer-readable medium for execution by, or to control the operation of, data processing apparatus. The computer readable-medium may be a machine-readable storage device, a machine-readable storage substrate, a memory device, a composition of matter affecting a machine-readable propagated signal, or a combination of one or more of them. The computer-readable medium may be a non-transitory computer-readable medium. The term “data processing apparatus” encompasses all apparatus, devices, and machines for processing data, including by way of example a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as a program, software, software application, script, or code) may be written in any form of programming language, including compiled or interpreted languages, and it may be deployed in any form, including as a standalone program or as a module, component, subroutine, or other unit suitable for use in a computing environment. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform functions by operating on input data and generating output. The processes and logic flows may also be performed by, and apparatus may also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer may be embedded in another device, e.g., a tablet computer, a mobile telephone, a personal digital assistant (PDA), a mobile audio player, a Global Positioning System (GPS) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory may be supplemented by, or incorporated in, special purpose logic circuitry.

To provide for interaction with a user, the techniques disclosed may be implemented on a computer having a display device, e.g., a CRT (cathode ray tube) or LCD (liquid crystal display) monitor, for displaying information to the user and a keyboard and a pointing device, e.g., a mouse or a trackball, by which the user may provide input to the computer. Other kinds of devices may be used to provide for interaction with a user as well; for example, feedback provided to the user may be any form of sensory feedback, e.g., visual feedback, auditory feedback, or tactile feedback; and input from the user may be received in any form, including acoustic, speech, or tactile input.

Implementations may include a computing system that includes a back end component, e.g., as a data server, or that includes a middleware component, e.g., an application server, or that includes a front end component, e.g., a client computer having a graphical user interface or a Web browser through which a user may interact with an implementation of the techniques disclosed, or any combination of one or more such back end, middleware, or front end components. The components of the system may be interconnected by any form or medium of digital data communication, e.g., a communication network. Examples of communication networks include a local area network (“LAN”) and a wide area network (“WAN”), e.g., the Internet.

The computing system may include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

Thus, particular implementations have been described. Other implementations are within the scope of the following claims. For example, the actions recited in the claims may be performed in a different order and still achieve desirable results.

What is claimed is:

1. A transcranial ultrasonic stimulation headset, comprising:
a coupling system;
one or more ultrasound transducers configured to generate and direct a first focused ultrasound beam at a region within a portion of a subject's brain;
one or more sensors configured to measure a response from the portion of the subject's brain in response to the first focused ultrasound beam; and
an electronic controller in communication with the one or more ultrasound transducers configured to dynamically adjust, based on the measured response from the portion of the subject's brain, a particular stimulation parameter for the one or more ultrasound transducers to generate and direct a second focused ultrasound beam at the region within a portion of the subject's brain.

2. The system of claim 1, further comprising an insert having a particular material property, and
wherein the electronic controller is further configured to adjust the particular material property of the insert based on the adjusted stimulation parameter.

3. The system of claim 2, wherein the insert is shaped based on a physical structure of the subject's skull.

4. The system of claim 1, wherein dynamically adjusting the stimulation parameter is performed based on the subject's verbal feedback.

5. The system of claim 1, wherein the coupling system comprises a cooling system having cooling fluid, and
wherein the coupling system is integrated with a neck support structure.

6. The system of claim 5, wherein the cooling fluid has a material property, and
wherein the electronic controller is further configured to adjust the material property of the insert based on the adjusted stimulation parameter.

7. The system of claim 6, wherein the material property is a density.

8. The system of claim 1, wherein the electronic controller is further configured to dynamically adjust two more stimulation parameters for the one or more ultrasound transducers.

9. A method for transcranial stimulation comprising:
directing, by one or more ultrasound transducers, a first focused ultrasound beam at a region within a portion of a subject's brain;
measuring, by one or more sensors and in response to the first focused ultrasound beam, a response from the subject's brain;
dynamically adjusting, based on the measured response from the portion of the subject's brain and by a controller, a stimulation parameter for the one or more ultrasound transducers to generate and direct a second focused ultrasound beam at the region within the portion of the subject's brain.

10. The method of claim 9, further comprising:
adjusting, by the controller and based on the adjusted stimulation parameter, a particular material property of an insert of a coupling system communicatively connected to the controller.

11. The method of claim 9, wherein measuring a response from the subject's brain comprises:
identifying an activity pattern of a subject's brain; and
determining, based on the identified activity pattern of the subject's brain and a target parameter, one or more stimulation parameters.

12. The method of claim 11, wherein the target parameter is a selected set of one or more physiological measurements of the subject.

13. The method of claim 11, wherein the target parameter is determined based on the subject's verbal feedback.

14. The method of claim 9, wherein dynamically adjusting the particular stimulation parameter is performed based on the subject's verbal feedback.

15. The method of claim 9, wherein dynamically adjusting the particular stimulation parameter comprises using machine learning techniques to generate one or more adjusted stimulation parameters.

16. The method of claim 9, wherein dynamically adjusting the particular stimulation parameter comprises adjusting two more stimulation parameters for the one or more ultrasound transducers.

17. A non-transitory computer storage medium encoded with instructions that when executed by a distributed computing system cause the distributed computing system to perform operations comprising:
directing, by one or more ultrasound transducers, a first focused ultrasound beam at a region within a portion of a subject's brain;
measuring, by one or more sensors and in response to the first focused ultrasound beam, a response from the subject's brain;
dynamically adjusting, based on the measured response from the portion of the subject's brain and by a controller, a stimulation parameter for the one or more ultrasound transducers to generate and direct a second focused ultrasound beam at the region within the portion of the subject's brain.

18. The non-transitory computer storage medium of claim 17, the operations further comprising:
adjusting, by the controller and based on the adjusted stimulation parameter, a particular material property of an insert of a coupling system communicatively connected to the controller.

19. The non-transitory computer storage medium of claim 17, wherein measuring a response from the subject's brain comprises:
identifying an activity pattern of a subject's brain; and
determining, based on the identified activity pattern of the subject's brain and a target parameter, one or more stimulation parameters.

20. The non-transitory computer storage medium of claim 19, wherein the target parameter is a selected set of one or more physiological measurements of the subject.
